(19) United States
(12) Patent Application Publication
(10) Pub. No.: US 2015/0305950 A1
(43) Pub. Date: Oct. 29, 2015

(54) TAMPON WRAPPER

(71) Applicant: Kimberly-Clark Worldwide, Inc., Neenah, WI (US)

(72) Inventors: Zachary D. Cousineau, Kaukauna, WI (US); Martin Gottvald, Appleton, WI (US); Gregg M. Brey, Oshkosh, WI (US); Randall William Foss, Appleton, WI (US); Charles Robert Tomsovic, Omro, WI (US); Steven Craig Gehling, Oshkosh, WI (US)

(73) Assignee: Kimberly-Clark Worldwide, Inc., Neenah, WI (US)

(21) Appl. No.: 14/263,662

(22) Filed: Apr. 28, 2014

(51) Int. Cl. A61F 13/551

(52) U.S. Cl. CPC A61F 13/55175

(57) ABSTRACT

A wrapping material for a tampon which can reduce or prevent contamination of the tampon and/or can reduce or prevent loss of a treatment agent from the tampon. A tampon wrapped in a wrapping material can experience a reduction in the contamination of the tampon and/or a reduction in the loss of a treatment agent from the tampon.

### BACKGROUND

Personal care articles are generally packaged into wrapping material which can be easily opened by the user of the personal care article. While the wrapping material can be easily opened, it should also protect the personal care article from dirt, dust, moisture, microorganisms or other contamination. Particular care should be taken to prevent contamination of those personal care articles which are intended to be inserted into the body of the user. For personal care articles which have been treated with a lotion, a fragrance, a medicament or other treatment agent, the wrapping material should also protect these treatment agents and prevent loss of the treatment agent through migration of the treatment agent through the wrapping material and away from the personal care article.

In order to prevent contamination and/or prevent loss of a treatment agent, personal care articles, such as tampons which are meant to be inserted digitally as opposed to insertion via an applicator, have employed tight wrappers wherein each tampon is heat sealed within a tubular sleeve of wrapping material. The wrapping material can serve at least three functions. The first function can be to prevent dirt or other foreign material from contaminating the tampon prior to use. The second function can be to maintain the presence of a treatment agent within the tampon and prevent migration of the treatment agent through the wrapping material and away from the tampon. Thirdly, as products such as tampons generally comprise absorbent material, they tend to soak up ambient moisture and deform. The third function of the wrapper, therefore, can be to maintain the shape of the tampon and prevent deformation.

The wrapper of a tampon, can be tightly wrapped around the tampon which can, in turn, increase the frictional forces existing between the tampon and the inside surface of the wrapper. An increase in the frictional forces can result in a wrapper which can be difficult to remove from around the tampon. To provide a mechanism which can allow for easier removal of the wrapper from around the tampon, the wrapping material can be weakened, such as, for example, through the use of a line of weakness. When opening the wrapper of the tampon, the line of weakness can be manipulated by the user of the tampon which can provide an easier opening to the tampon.

The line of weakness, however, can provide a pathway for dirt, dust, moisture, microorganisms or other contamination to enter the tampon or a treatment agent to migrate away from a tampon. There remains a need for a wrapper for a tampon which can be easily opened by a user and which does not expose the tampon to contamination and/or expose the tampon to loss of a treatment agent. There remains a need for a wrapping material for a tampon which can withstand the radial force exerted on the wrapping material by way of the natural expansion of the tampon but which can give way to the stress of shear forces and longitudinal bending forces which can be exerted on the wrapping material by way of a user of the tampon opening the wrapping material to gain access to the tampon.

### SUMMARY

In various embodiments, a wrapping material for a tampon can comprise, in a laid-flat configuration, a longitudinal direction, a lateral direction, a first lateral edge, a second lateral edge, a zone of overlap and a line of weakness located within the zone of overlap. In various embodiments, the zone of overlap can further comprise a first fold and a second fold. In various embodiments, the line of weakness can be located between the first and second folds. In various embodiments, the line of weakness can be located between one of the first or second folds and one of the first or second lateral side edges. In various embodiments, the wrapping material can be cellophane. In various embodiments, the wrapping material can comprise two pieces of material bonded together. In various embodiments, the wrapping material can comprise a single layer of material.

In various embodiments, a wrapped tampon comprising a tampon and a wrapping material, wherein the wrapping material comprises a zone of overlap and a line of weakness associated with the zone of overlap. In various embodiments, the zone of overlap can be a z-fold configuration comprising an inner layer, an outer layer, and a middle layer located between the inner layer and the outer layer. In various embodiments, the line of weakness can be located on the inner layer of the z-fold configuration of the zone of overlap. In various embodiments, the line of weakness can be located on the outer layer of the z-fold configuration of the zone of overlap. In various embodiments, the line of weakness can be located on the middle layer of the z-fold configuration of the zone of overlap. In various embodiments, the zone of overlap can encircle the circumference of the tampon. In various embodiments, the zone of overlap can encircle the tampon in an insertion zone of the tampon. In various embodiments, the zone of overlap can encircle the tampon in a withdrawal zone of the tampon. In various embodiments, the zone of overlap can encircle the tampon in an intermediate zone of the tampon. In various embodiments, the zone of overlap can encircle the tampon at least partially in an insertion zone and at least partially in an intermediate zone of the tampon. In various embodiments, the zone of overlap can encircle the tampon at least partially in a withdrawal zone and at least partially in an intermediate zone of the tampon. In various embodiments, the wrapping material can comprise a single layer of material. In various embodiments, the wrapping material can comprise two pieces of material bonded together.

### BRIEF DESCRIPTION OF THE DRAWINGS

FIG. 1 is a side view of an embodiment of a wrapped tampon.

FIGS. 2-4 are planar views of various embodiments of a wrapping material.

FIG. 5 is a side view of a longitudinal cross-section of an embodiment of a wrapped tampon wherein the inside of the tampon is facing the viewer.

FIGS. 5A-5F are enlarged schematic views of various embodiments of a zone of overlap of the wrapping material illustrated in FIG. 5.

FIG. 6 is a planar view of an embodiment of a wrapping material.

FIGS. 7A and 7B are schematic views of embodiments of a zone of overlap of the wrapping material of FIG. 6.

### DETAILED DESCRIPTION

The present disclosure is generally directed towards a wrapping material for a tampon. The wrapping material can reduce or prevent contamination of the tampon and/or can reduce or prevent loss of a treatment agent from the tampon. The present disclosure is also generally directed towards a tampon wrapped in a wrapping material which can experience a reduction in the contamination of the tampon and/or a reduction in the loss of a treatment agent from the tampon.

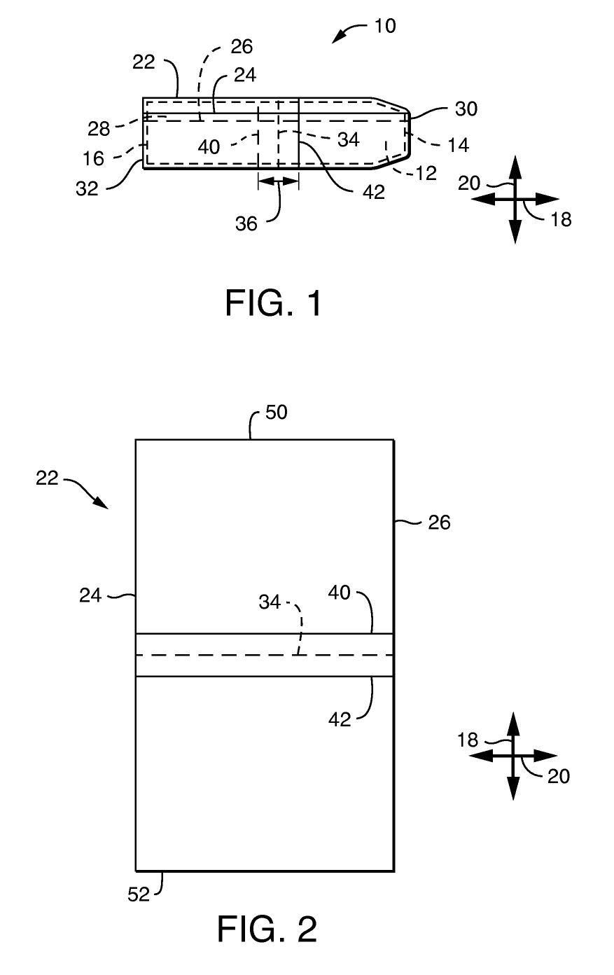

Referring to the Figures, illustrated in FIG. 1 is a wrapped absorbent article 10 such as a tampon 12. The tampon 12 can be composed of any material deemed suitable, can be compressed in any manner known to one of ordinary skill, and formed into any desired shape or configuration deemed to be suitable. The tampon 12 can have an insertion end 14 and a withdrawal end 16. The longitudinal length of the tampon 12, in the longitudinal direction 18, can be any length as deemed suitable. The compressed width of the tampon 12, in the lateral direction 20, can be any width as deemed suitable and can be uniform or can vary when moving in the longitudinal direction 18 from the insertion end 14 to the withdrawal end 16.

As illustrated in FIG. 1, the tampon 12 can be wrapped in a wrapping material 22. The wrapping material 22 can be chosen from a wide variety of commonly used wrapping materials 22 such as polymeric films, metal foils, or treated papers. In various embodiments, the wrapping material 22 can be cellophane. Cellophane can provide a variety of desired physical attributes such as, but not limited to, strength, flexibility and tearability. In various embodiments, the cellophane can be treated such as by being treated with a coating to be heat sealable.

In various embodiments, the wrapping material 22 can be a single layer of material or can be a laminate of two or more materials which can be combined into a single layer of wrapping material 22. In various embodiments, the wrapping material 22 can be formed from two pieces of material which can be bonded together, such as, for example, by bonding a lateral edge of one piece of material to a lateral edge of the other piece of material, to form a single wrapping material 22. The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. The bonding can occur via adhesive, pressure bonding, thermal bonding, ultrasonic bonding, and/or welding, or any other means deemed suitable.

The wrapping material 22, whether a single layer of material, a laminate of two or more materials combined into a single layer, or two pieces which have been bonded together to form a single wrapping material 22, can have longitudinal side edges, 24 and 26, and when the wrapping material 22 is wrapped around the tampon 12, one of the longitudinal side edges, such as longitudinal side edge 24, can overlap the other longitudinal side edge, such as longitudinal side edge 26, to produce a longitudinal seam 28. The amount of overlap (i.e., the width of the longitudinal seam 28 in the lateral direction 20) can be any amount deemed suitable to provide a longitudinal seam 28 which can remain in a sealed configuration until disrupted by a user of the tampon 12. As illustrated in FIG. 1, the insertion end 14 and the withdrawal end 16 of the tampon 12 may be enclosed by the wrapping material 22. The ends, 30 and 32, of the wrapping material 22 can be closed about the insertion end 14 and the withdrawal end 16 of the tampon 12 by any means known to one of ordinary skill in the art such as, for example, thermal bonding, adhesives, twisting, folding or crimping closed.

To provide a user with a wrapped tampon 12 which can be easy to open, the wrapping material 22, whether a single layer of material, a laminate of two or more materials combined into a single layer, or two pieces which have been bonded together to form a single wrapping material 22, can be provided with a line of weakness 34. The line of weakness 34 can be a line which can run through the wrapping material 22 and can be composed of a row of weak points which can be uniform, or different, in size and type, so that the wrapping material 22 can comprise two parts connected by the line of weakness 34. The line of weakness 34 can also be composed of a row of weak points which can have any desired geometry, such as, for example, arrows, semi-circles, circles, rectangles, etc. The line of weakness 34 can be linear, curvy, wavy, zig-zag or any other configuration as deemed suitable. The stability of the line of weakness 34 can be matched to the desired material properties of the wrapping material 22. Matching the stability of the line of weakness 34 to the material properties, such as, for example, the flexibility, of the wrapping material 22 can provide for a wrapped tampon 12 wherein the user does not have to expend any great force to destroy the line of weakness 34. Additionally, matching the stability of the line of weakness 34 to the material properties of the wrapping material 22 can avoid inadvertent destruction of the line of weakness 34. In various embodiments, the line of weakness 34 can be formed by perforations and/or a reduced material thickness in the area of the line of weakness 34. In various embodiments such as when the wrapping material 22 can be formed by bonding two pieces of material together, the line of weakness 34 can be spaced apart, discrete areas of bonding, such as, for example, but not limited to, adhesive, ultrasonic or thermal bond points, etc. In various embodiments, more than one line of weakness 34 can be present in the wrapping material 22. For example, at least two lines of weakness 34 can be present in the wrapping material 22. Each line of weakness 34 can at least partially encircle the circumference of the tampon 12, however, each line of weakness 34 need not fully encircle the tampon 12. In such an embodiment, each line of weakness 34 can be offset from each other line of weakness 34 in the longitudinal direction 18 as the wrapping material 22 encircles the tampon 12. In various embodiments, the line of weakness 34 can be present in a non-uniform distribution, such as, for example, the line of weakness 34 can have a non-uniform strength relative to either shear or radial expansion stresses. For example, a tampon 12 can be compressed in such a manner that the tampon 12 may naturally attempt to expand in a non-uniform manner. The line of weakness 34 of the wrapping material 22 can be arranged as deemed suitable to prevent breakage of the line of weakness 34 following any expansion of the tampon 12.

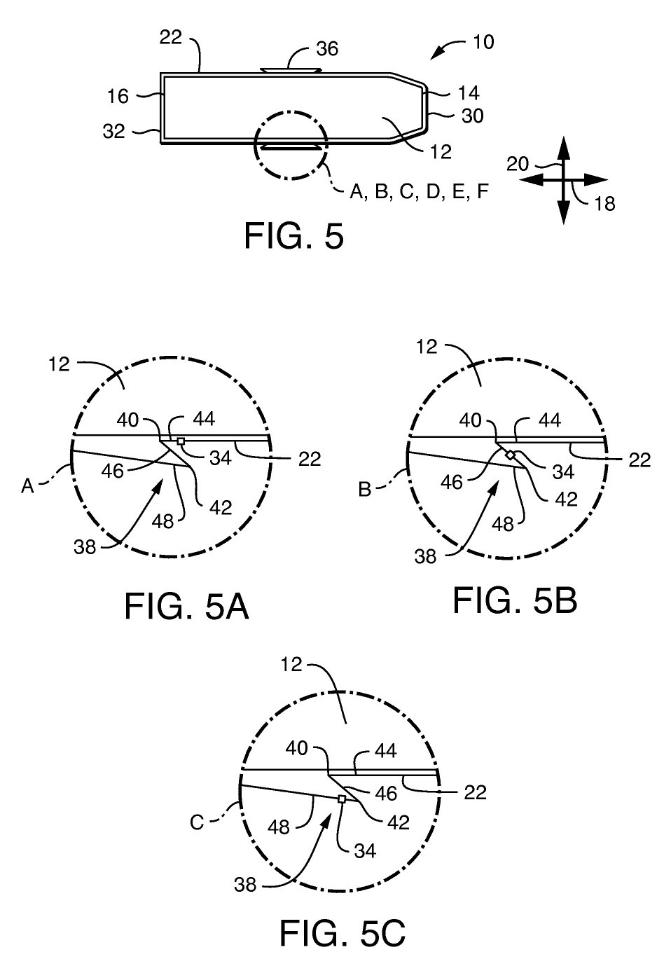

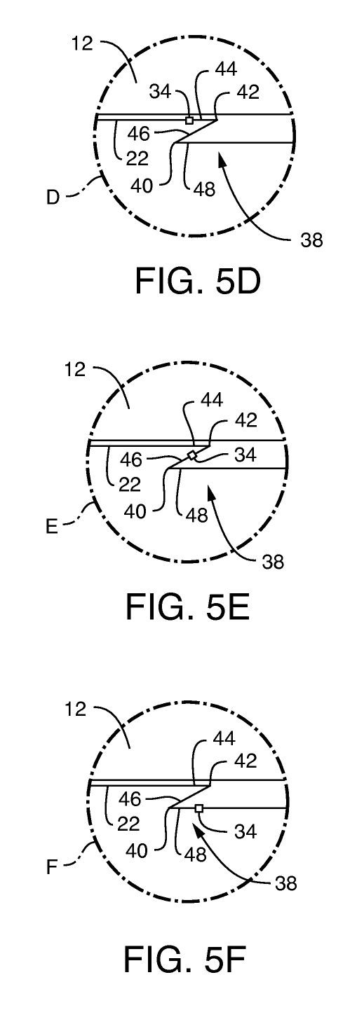

To protect the tampon 12 within the wrapping material 22 from dirt, dust, moisture, microorganisms, or other contamination which may enter through the line of weakness 34 and/or to prevent the loss of a treatment agent which may exit through the line of weakness 34, a zone of overlap 36 can be created in the wrapping material 22, whether a single layer of material, a laminate of two or more materials combined into a single layer, or two pieces which have been bonded together to form a single wrapping material 22. The zone of overlap 36 can be a location of the wrapping material 22 wherein a portion of the wrapping material 22 can overlap another portion of the wrapping material 22. The zone of overlap 36 can encircle the tampon 12 at any desired location of the wrapped tampon 12 as will be described herein. It is to be understood that the zone of overlap 36 can be created by a selective folding and overlapping of the wrapping material 22 rather than fully encircling and enclosing the tampon 12 in two or more layers of wrapping material 22 for the entire longitudinal length of the tampon 12. In various embodiments, the zone of overlap 36 can be a location of the wrapping material 22 wherein a first portion of the wrapping material 22 can be folded over a second portion of the wrapping material 22 creating at least two layers of wrapping material 22 in the zone of overlap 36. In various embodiments, the zone of overlap 36 can be a location of the wrapping material 22 wherein a first portion 22 of the wrapping material 22 can be folded over a second portion of the wrapping material 22 and a third portion of the wrapping material 22 can be folded over the first portion of the wrapping material 22 creating at least three layers of wrapping material 22 in the zone of overlap 36. Such a folded configuration can resemble a z-fold configuration 38 in the wrapping material 22 (such as illustrated in FIGS. 5A-5F, 7A and 7B).

When a portion of the wrapping material 22 is folded over another portion of the wrapping material 22, a fold can be created in the wrapping material 22. FIG. 1 provides an illustration of two folds, 40 and 42, in the wrapping material 22. In the illustration of FIG. 1, fold 40 underlays a portion of the wrapping material 22 and is adjacent to the tampon 12 while fold 42 is on an external surface of the wrapped article 10 overlaying a portion of the wrapping material 22 and is not adjacent to the tampon 12. It is to be understood that the configuration can be reversed and fold 42 could underlie a portion of the wrapping material 22 and be adjacent to the tampon 12 and fold 40 could be on an external surface of the wrapped article 10 overlaying a portion of the wrapping material 22 and not adjacent to the tampon 12.

The wrapping material 22 can be tightly wound about the tampon 12 and can, therefore, have substantially the same dimensions, in the wrapped configuration, as the tampon 12. In various embodiments, the zone of overlap 36 can be located at substantially the midpoint of the longitudinal length of the wrapping material 22 in the wrapped configuration about the wrapped article 10 (such as illustrated in FIG. 1). In various embodiments, the zone of overlap 36 of the wrapping material 22, in a wrapped configuration about a wrapped article 10, can be positioned encircling the wrapped article 10 at a location between the insertion end 14 and the midpoint of the longitudinal length of the wrapped article 10. In various embodiments, the zone of overlap 36 of the wrapping material 22, in a wrapped configuration about a wrapped article 10, can be positioned encircling the wrapped article 10 at a location between the withdrawal end 16 and the midpoint of the longitudinal length of the wrapped article 10.

In various embodiments, the longitudinal length of the tampon 12 can be divided into zones, such as an insertion zone which will enter the body first and which can be about one-third of the longitudinal length of the tampon 12, a withdrawal zone which will enter the body last and can be about one-third of the longitudinal length of the tampon 12, and an intermediate zone which can be about one-third of the longitudinal length between the insertion zone and the withdrawal zone. In various embodiments, the zone of overlap 36 of the wrapping material 22, in a wrapped configuration about a tampon 12, can be positioned at least partially encircling the insertion zone of the tampon 12. In various embodiments, the zone of overlap 36 of the wrapping material 22, in a wrapped configuration about a tampon 12, can be positioned at least partially encircling the withdrawal zone of the tampon 12. In various embodiments, the zone of overlap 36 of the wrapping material 22, in a wrapped configuration about a tampon 12, can be positioned at least partially encircling the intermediate zone of the tampon 12. In various embodiments, the zone of overlap 36 of the wrapping material 22, in a wrapped configuration about a tampon 12, can be positioned at least partially encircling the insertion zone and at least partially encircling the intermediate zone of the tampon 12. In various embodiments, the zone of overlap 36 of the wrapping material 22, in a wrapped configuration about a tampon 12, can be positioned at least partially encircling the withdrawal zone and at least partially encircling the intermediate zone of the tampon 12.

In various embodiments, the zone of overlap 36 can have any longitudinal length as desired when in the folded configuration such as illustrated in FIG. 1. In various embodiments, the longitudinal length of the zone of overlap 36 in a folded configuration can range from about 1, 2, 3, 4, 5, 6 or 7 mm to about 8, 9, 10, 11, 12, 13, 14, or 15 mm. In various embodiments, the longitudinal length of the wrapping material 22 incorporated into the zone of overlap 36, when in a flat, non-folded configuration, can range from about 3, 4, 5, 6, 7, 8, 9, 10, 11, 12, 13, 14, 15, 16, 17, 18, 19, 20, 21, 22, 23 or 24 mm to about 25, 26, 27, 28, 29, 30, 31, 32, 33, 34, 35, 36, 36, 38, 39, 40, 41, 42, 43, 44 or 45 mm. As a non-limiting example, the longitudinal length of the wrapping material 22 incorporated into a zone of overlap 36, in a flat, non-folded configuration, can be about 21 mm and the resultant zone of overlap 36 with two folds, 40 and 42, and each overlapping layer being of substantially the same longitudinal length, can have a longitudinal length of about 7 mm.

To reduce contamination of the tampon 20 and/or to prevent loss of a treatment agent, the line of weakness 34 can be associated with the zone of overlap 36 and can be located on any portion and in any of the overlapping layers of wrapping material 22 incorporated into the zone of overlap 36. In various embodiments, the zone of overlap 36 can be folded into a z-fold configuration 38 and can have three layers of overlapping wrapping material 22 incorporated into the zone of overlap 36. The three layers of the overlapping wrapping material 22 can include an inner layer 44 located closest to the tampon 12, an outer layer 48 located furthest from the tampon 12, and a middle layer 46 located between the inner layer 44 and the outer layer 48. In various embodiments, the line of weakness 34 can be located on the inner layer 44 of the wrapping material 22 in the zone of overlap 36. In various embodiments, the line of weakness 34 can be located on the middle layer 46 of the wrapping material 22 in the zone of overlap 36. In various embodiments, the line of weakness 34 can be located on the outer layer 48 of the wrapping material 22 in the zone of overlap 36. In various embodiments, the line of weakness 34 can be located on fold 40 or fold 42 of the wrapping material 22. As the line of weakness 34 can be located in a portion of the wrapping material 22 which is overlapping another portion of the wrapping material 22, i.e., the zone of overlap 36, the pathway for dirt, dust, moisture, microorganisms or other contaminants and/or the pathway for migration outward of a treatment agent can be obstructed and can reduce and/or eliminate the contamination of the tampon 12 and/or reduce or eliminate the loss of a treatment agent associated with the tampon 12 within the wrapping material 22.

In various embodiments, the zone of overlap 36 can remain in the folded configuration about the tampon 12 by utilizing the expansion capability of the tampon 12 which can increase the forces exerted upon the wrapping material 22. A tampon 12 can be composed of a highly compressed absorbent material. The absorbent material can have a natural tendency to rebound from the compression undergone during the manufacturing process. The rebounding of the tampon 12 from a highly compressed configuration in the manufacturing process to a slightly less compressed configuration in the wrapping material 22 can direct the expansion forces of the absorbent material of the tampon 12 in a direction towards the wrapping material 22 which can push the overlapping layers of the wrapping material 22 together and maintain the zone of overlap 36.

In various embodiments, the zone of overlap 36 can remain in the folded configuration by bonding at least two of the layers of wrapping material 22 together such as, for example, but not limited to, through ultrasonic bonding, thermal bonding or adhesives or any other means deemed suitable. The bonding of the at least two layers of wrapping material 22 together can occur along the full circumference of the zone of overlap 36 around the tampon 12 or can occur intermittently along the circumference of the zone of overlap 36 around the tampon 12. In various embodiments, the bonding of the at least two layers of wrapping material 22 can occur in a single location of the zone of overlap 36. In various embodiments, the inner layer 44 and the middle layer 46 can be bonded together. In various embodiments, the middle layer 46 and the outer layer 48 can be bonded together. In various embodiments, the inner layer 44 and the middle layer 46 can be bonded together and the middle layer 46 and the outer layer 48 can be bonded together.

To open the wrapping material, the user of the tampon 12 can grasp the wrapped article 10 at the insertion end 14 with one hand and at the withdrawal end 16 with the other hand and twist the wrapping material 22 in opposite directions. The twisting motion can disrupt the line of weakness 34 and the user can separate the resultant two pieces of wrapping material 22 from each other. In various embodiments, indices can be incorporated on the wrapping material 22 to provide notice to the user as to locations where to grasp the wrapped article 10. Such indices can include, but are not limited to, text, graphics, changes in surface topography of the wrapping material 22, and inclusion of a surface treatment to change increase the adhesion or friction of the wrapping material 22 to a user's fingers.

Referring to FIGS. 2-4, the wrapping material 22 in a flat, non-folded configuration can be illustrated. In each of the illustrations of FIGS. 2-4, the wrapping material 22 can be a single layer of material or can be a laminate of two or more pieces of material into a single layer of wrapping material 22. In each of the illustrations of FIGS. 2-4, the wrapping material 22 can have longitudinal side edges, 24 and 26, and lateral edges, 50 and 52. As described above, when the wrapping material 22 is wrapped about a tampon 12, a longitudinal side edge, such as longitudinal side edge 24 can overlap the other longitudinal side edge, such as longitudinal side edge 26, to create the longitudinal seam 28 (as illustrated in FIG. 1). The lateral edges, 50 and 52, can form part of the end portions of the wrapping material 22 which can enclose the insertion end 14 and the withdrawal end 16 of the tampon 12. In various embodiments, lateral edge 50 can be located near the insertion end 14 of the tampon 12 and lateral edge 52 can be located near the withdrawal end 16 of the tampon 12 when the wrapping material 22 is wrapped about the tampon 12. It is to be understood that the lateral edges can be reversed and lateral edge 50 can be located near the withdrawal end 16 of the tampon 12 and lateral edge 52 can be located near the insertion end 14. Also illustrated in FIGS. 2-4 is a line of weakness 34 which can be incorporated into the wrapping material 22.

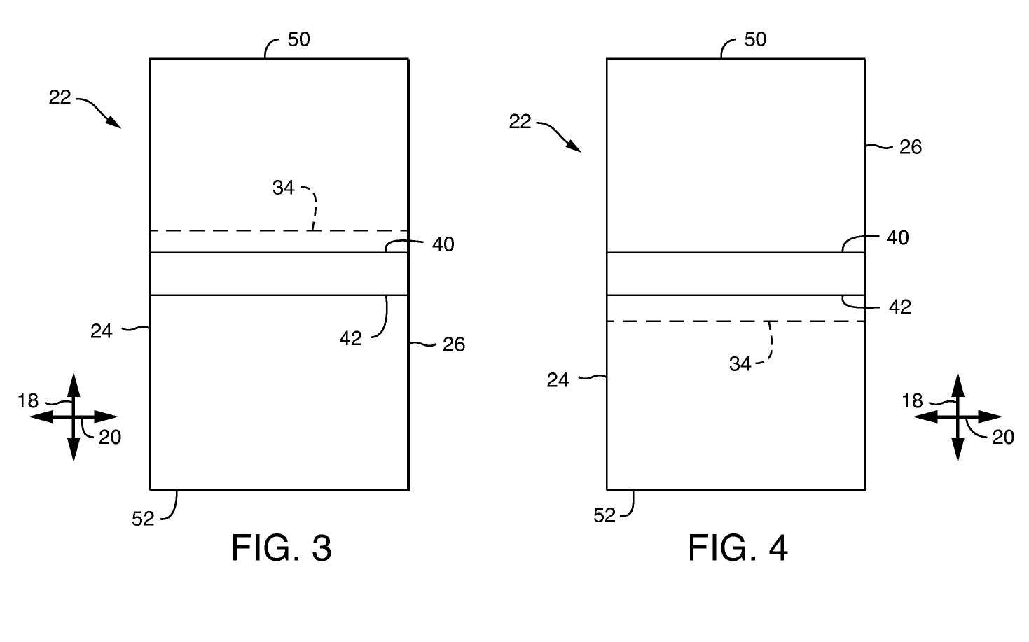

The wrapping material 22 can be configured to have a zone of overlap 36 prior to wrapping the wrapping material 22 about the tampon 12. As described above, the zone of overlap 36 can include at least two folds, such as folds 40 and 42. FIGS. 2-4 provide illustrations of various embodiments of where a line of weakness 34 can be located with respect to the folds, 40 and 42, which will be incorporated into the wrapping material 22 upon formation of the zone of overlap 36. As illustrated in FIG. 2, in an embodiment, a line of weakness 34 can be located between the portions of wrapping material 22 which will become folds, 40 and 42, during formation of the zone of overlap 36. In such an embodiment, the line of weakness 34 can be positioned on the resultant middle layer 46 of the zone of overlap 36. As illustrated in FIG. 3, in an embodiment, a line of weakness 34 can be located between lateral edge 50 and the portion of the wrapping material 22 which will become fold 40 during formation of the zone of overlap 36. The line of weakness 34 can be located any distance from the portion of the wrapping material 22 which will become fold 40 during formation of the zone of overlap 36 as long as the line of weakness 34 will be incorporated into the zone of overlap 36. In the embodiment illustrated in FIG. 3, the line of weakness 34 can be positioned on the resultant inner layer 44 of the zone of overlap 36 in embodiments wherein fold 40 underlays the wrapping material 22 and is located adjacent the tampon 12. In the embodiment illustrated in FIG. 3, the line of weakness 34 can be positioned on the resultant outer layer 48 of the zone of overlap 36 in embodiments wherein fold 40 is located on the external surface of the wrapping material 22 in the zone of overlap 36. As illustrated in FIG. 4, in an embodiment, a line of weakness 34 can be located between lateral edge 52 and the portion of the wrapping material 22 which will become fold 42 during formation of the zone of overlap 36. The line of weakness 34 can be located any distance from the portion of the wrapping material 22 which will become fold 42 during formation of the zone of overlap 36 as long as the line of weakness 34 will be incorporated into the zone of overlap 36. In the embodiment illustrated in FIG. 4, the line of weakness can be positioned on the resultant inner layer 44 of the zone of overlap 36 in embodiments wherein fold 42 underlays the wrapping material 22 and is located adjacent the tampon 12. In the embodiment illustrated in FIG. 4, the line of weakness 34 can be positioned on the resultant outer layer 48 of the zone of overlap 36 in embodiments wherein fold 42 is located on the external surface of the wrapping material 22 in the zone of overlap 36.

FIG. 5 provides a side view of a longitudinal cross-section of an embodiment of a wrapped tampon 12 wherein the inside of the tampon 12 is facing the viewer. A zone of overlap 36 can be illustrated in the wrapping material 22 enclosing the wrapped tampon 12. FIGS. 5A-5F provide close-up schematic illustrations of various embodiments of the zone of overlap 36 of the wrapping material 22 as related to the wrapped tampon 12 of FIG. 5. FIG. 5A provides a close-up schematic illustration of a zone of overlap 36 having a z-fold configuration 38. In the z-fold configuration, fold 40 underlays the wrapping material 22 and can be positioned adjacent the tampon 12. Fold 42 can be on the external surface of the wrapping material 22. In the embodiment illustrated in FIG. 5A, the line of weakness 34 can be located on the inner layer 44 of the zone of overlap 36. In the embodiment illustrated, the external fold 42 is pointing in the direction of the insertion end 14 of the tampon 12 illustrated in FIG. 5. FIG. 5B provides a close-up schematic illustration of a zone of overlap 36 having a z-fold configuration 38. In the z-fold configuration, fold 40 underlays the wrapping material 22 and can be positioned adjacent the tampon 12. Fold 42 can be on the external surface of the wrapping material 22. In the embodiment illustrated in FIG. 5B, the line of weakness 34 can be located on the middle layer 46 of zone of overlap 36. In the embodiment illustrated, the external fold 42 is pointing in the direction of the insertion end 14 of the tampon 12 illustrated in FIG. 5. FIG. 5C provides a close-up schematic illustration of a zone of overlap 36 having a z-fold configuration 38. In the z-fold configuration, fold 40 underlays the wrapping material 22 and can be positioned adjacent the tampon 12. Fold 42 can be on the external surface of the wrapping material 22. In the embodiment illustrated in FIG. 5C, the line of weakness 34 can be located on the outer layer 48 of zone of overlap 36. In the embodiment illustrated, the external fold 42 is pointing in the direction of the insertion end 14 of the tampon 12 illustrated in FIG. 5. FIG. 5D provides a close-up schematic illustration of a zone of overlap 36 having a z-fold configuration 38. In the z-fold configuration, fold 42 underlays the wrapping material 22 and can be positioned adjacent the tampon 12. Fold 40 can be on the external surface of the wrapping material 22. In the embodiment illustrated in FIG. 5D, the line of weakness 34 can be located on the inner layer 44 of zone of overlap 36. In the embodiment illustrated, the external fold 40 is pointing in the direction of the withdrawal end 16 of the tampon 12 illustrated in FIG. 5. FIG. 5E provides a close-up schematic illustration of a zone of overlap 36 having a z-fold configuration 38. In the z-fold configuration, fold 42 underlays the wrapping material 22 and can be positioned adjacent the tampon 12. Fold 40 can be on the external surface of the wrapping material 22. In the embodiment illustrated in FIG. 5E, the line of weakness 34 can be located on the middle layer 46 of zone of overlap 36. In the embodiment illustrated, the external fold 40 is pointing in the direction of the withdrawal end 16 of the tampon 12 illustrated in FIG. 5. FIG. 5F provides a close-up schematic illustration of a zone of overlap 36 having a z-fold configuration 38. In the z-fold configuration, fold 42 underlays the wrapping material 22 and can be positioned adjacent the tampon 12. Fold 40 can be on the external surface of the wrapping material 22. In the embodiment illustrated in FIG. 5F, the line of weakness 34 can be located on the outer layer 48 of zone of overlap 36. In the embodiment illustrated, the external fold 40 is pointing in the direction of the withdrawal end 16 of the tampon 12 illustrated in FIG. 5. As described herein, in various embodiments, the line of weakness can be located on either of the folds, 40 or 42.

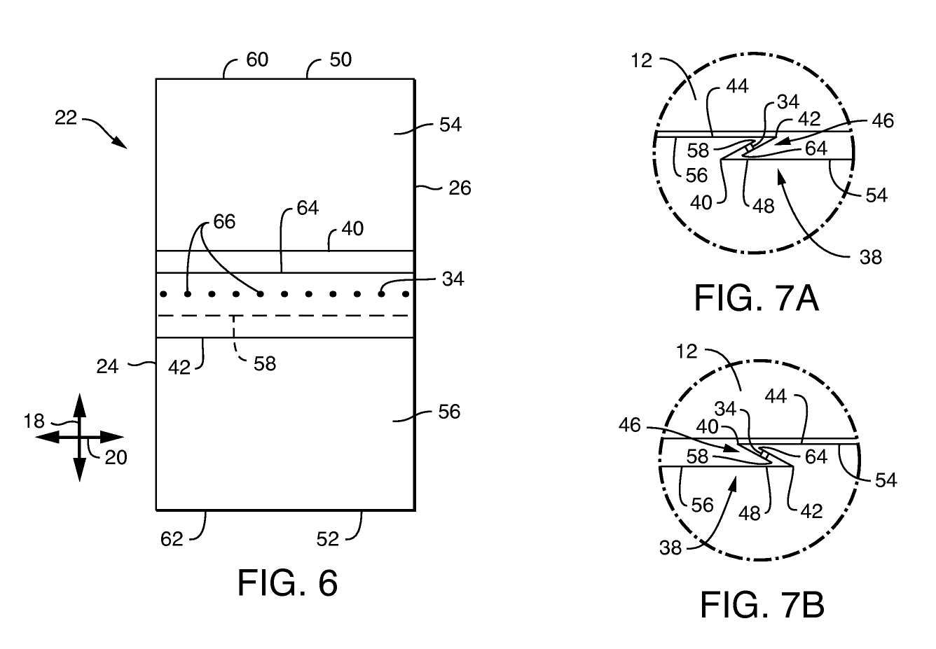

Referring to FIG. 6, the wrapping material 22 in a flat, non-folded configuration can be illustrated. In the illustration of FIG. 6, the wrapping material 22 can be two pieces of material, 54 and 56, which can be bonded together at a lateral edge of each of the pieces of material 54 and 56. In the illustration of FIG. 6, material 54 can have lateral edges 58 and 60 and material 56 can have lateral edges 62 and 64. To form the wrapping material 22, the two pieces of material 54 and 56 can be brought into an overlapping configuration and can be bonded together in the proximity of lateral edge 58 of material 54 and lateral edge 64 of material 56. The bonding of the two pieces of material 54 and 56 together can be continuous or intermittent. In various embodiments, the bonding of the materials 54 and 56 together can be intermittent and the discrete bond points 66 can form the line of weakness 34. The lateral edge 60 of material 54 can form lateral edge 50 of the wrapping material 22 and lateral edge 62 of material 56 can form lateral edge 52 of the wrapping material 22. Each piece of material 54 and 56 can have longitudinal side edges which can form the longitudinal side edges 24 and 26 of the wrapping material.

As described above, when the wrapping material 22 is wrapped about a tampon 12, a longitudinal side edge, such as longitudinal side edge 24 can overlap the other longitudinal side edge, such as longitudinal side edge 26, to create the longitudinal seam 28 (as illustrated in FIG. 1). The lateral edges, 50 and 52, can form part of the end portions of the wrapping material 22 which can enclose the insertion end 14 and the withdrawal end 16 of the tampon 12. In various embodiments, lateral edge 50 can be located near the insertion end 14 of the tampon 12 and lateral edge 52 can be located near the withdrawal end 16 of the tampon 12 when the wrapping material 22 is wrapped about the tampon 12. It is to be understood that the lateral edges can be reversed and lateral edge 50 can be located near the withdrawal end 16 of the tampon 12 and lateral edge 52 can be located near the insertion end 14.

The wrapping material 22 can be configured to have a zone of overlap 36 prior to wrapping the wrapping material 22 about the tampon 12. As described above, the zone of overlap 36 can include at least two folds, such as folds 40 and 42. FIG. 6 provides an illustration of an embodiment of where the line of weakness 34 can be located with respect to the folds, 40 and 42, which will be incorporated into the wrapping material 22 upon formation of the zone of overlap 36. As illustrated in FIG. 6, in an embodiment, a line of weakness 34 can be located between the portions of wrapping material 22 which will become folds, 40 and 42, during formation of the zone of overlap 36. In such an embodiment, the line of weakness 34 can be positioned on the resultant middle layer 46 of the zone of overlap 36.

FIGS. 7A and 7B provide close-up schematic illustrations of various embodiments of the zone of overlap 36 of the wrapping material 22. FIG. 7A provides a close-up schematic illustration of a zone of overlap 36 having a z-fold configuration 38. In the z-fold configuration, fold 42 underlays the wrapping material 22 and can be positioned adjacent the tampon 12. Fold 40 can be on the external surface of the wrapping material 22. In the embodiment illustrated in FIG. 7A, the line of weakness 34 can be located on the middle layer 46 of the zone of overlap 36. In the embodiment illustrated in FIG. 7A, the overlapping portion of the two pieces of material 54 and 56 which form the wrapping material 22 can be positioned between folds 40 and 42. In such an embodiment, lateral edge 58 and lateral edge 64 can both be positioned on the middle layer 46 of the zone of overlap 36. FIG. 7B provides a close-up schematic illustration of a zone of overlap 36 having a z-fold configuration 38. In the z-fold configuration, fold 40 underlays the wrapping material 22 and can be positioned adjacent the tampon 12. Fold 42 can be on the external surface of the wrapping material 22. In the embodiment illustrated in FIG. 7B, the line of weakness 34 can be located on the middle layer 46 of zone of overlap 36. In the embodiment illustrated in FIG. 7B, the overlapping portion of the two pieces of material 54 and 56 which form the wrapping material 22 can be positioned between folds 40 and 42. In such an embodiment, lateral edge 58 and lateral edge 64 can both be positioned on the middle layer 46 of the zone of overlap 36.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wrapping material for a tampon comprising, in a laid-flat configuration, a longitudinal direction, a lateral direction, a first lateral edge, a second lateral edge, a zone of overlap and a line of weakness located within the zone of overlap.

2. The wrapping material of claim 1 wherein the zone of overlap further comprises a first fold and a second fold.

3. The wrapping material of claim 2 wherein the line of weakness is located between the first and second folds.

4. The wrapping material of claim 2 wherein the line of weakness is located between one of the first or second folds and one of the first or second lateral side edges.

5. The wrapping material of claim 1 wherein the wrapping material is cellophane.

6. The wrapping material of claim 1 wherein the wrapping material comprises two pieces of material bonded together.

7. The wrapping material of claim 1 wherein the wrapping material comprises a single layer of material.

8. A wrapped tampon comprising a tampon and a wrapping material, wherein the wrapping material comprises a zone of overlap and a line of weakness associated with the zone of overlap.

9. The wrapped tampon of claim 8 wherein the zone of overlap is a z-fold configuration comprising an inner layer, an outer layer, and a middle layer located between the inner layer and the outer layer.

10. The wrapped tampon of claim 9 wherein the line of weakness is located on the inner layer of the z-fold configuration of the zone of overlap.

11. The wrapped tampon of claim 9 wherein the line of weakness is located on the outer layer of the z-fold configuration of the zone of overlap.

12. The wrapped tampon of claim 9 wherein the line of weakness is located on the middle layer of the z-fold configuration of the zone of overlap.

13. The wrapped tampon of claim 8 wherein the zone of overlap encircles the circumference of the tampon.

14. The wrapped tampon of claim 8 wherein the zone of overlap encircles the tampon in an insertion zone of the tampon.

15. The wrapped tampon of claim 8 wherein the zone of overlap encircles the tampon in a withdrawal zone of the tampon.

16. The wrapped tampon of claim 8 wherein the zone of overlap encircles the tampon in an intermediate zone of the tampon.

17. The wrapped tampon of claim 8 wherein the zone of overlap encircles the tampon at least partially in an insertion zone and at least partially in an intermediate zone of the tampon.

18. The wrapped tampon of claim 8 wherein the zone of overlap encircles the tampon at least partially in a withdrawal zone and at least partially in an intermediate zone of the tampon.

19. The wrapped tampon of claim 8 wherein the wrapping material comprises a single layer of material.

20. The wrapped tampon of claim 8 wherein the wrapping material comprises two pieces of material bonded together.
